(19) United States
(12) Patent Application Publication
(10) Pub. No.: US 2005/0112199 A1
(43) Pub. Date: May 26, 2005

(54) Therapeutic regimens for administering drug combinations

(76) Inventors: Mahesh Padval, Waltham, MA (US); Peter Elliott, Marlboro, MA (US)

Correspondence Address: CLARK & ELBING LLP, 101 FEDERAL STREET, BOSTON, MA, 02110, US

(21) Appl. No.: 10/947,769

(22) Filed: Sep. 23, 2004

Related U.S. Application Data
(63) Continuation-in-part of application No. 10/947,455, filed on Sep. 20, 2004.
Application No. 10/947,455 is a continuation of application No. 10/777,517, filed on Feb. 12, 2004.
Application No. 10/777,517 is a continuation-in-part of application No. 10/670,488, filed on Sep. 24, 2003.
Application No. 10/947,455 is a continuation-in-part of application No. 10/944,574, filed on Sep. 17, 2004.
Application No. 10/947,455 is a continuation-in-part of application No. 10/777,518, filed on Feb. 12, 2004.
(60) Provisional application No. 60/520,446, filed on Nov. 13, 2003.
(60) Provisional application No. 60/512,415, filed on Oct. 15, 2003.
(60) Provisional application No. 60/557,496, filed on Mar. 30, 2004.

(51) Int. Cl. A61K 9/22

(52) U.S. Cl. 424/468

(57) ABSTRACT

The invention features dosing regimens for the administration of combination therapies, wherein one of the drugs of the combination is formulated for sustained release, or administered repeatedly, and compositions related thereto.

### CROSS-REFERENCE TO RELATED APPLICATIONS

This application is Continuation-in-part of U.S. Utility application entitled “Methods and Reagents for the Treatment of Diseases and Disorders Associated with Increased Levels of Proinflammatory Cytokines,” filed Sep. 22, 2004, which is a Continuation of U.S. Utility application Ser. No. 10/777,517, filed Feb. 12, 2004, which is a Continuation-in-part of U.S. Utility application Ser. No. 10/670,488, filed Sep. 24, 2003, and claims benefit of U.S. Provisional Application No. 60/557,496, filed Mar. 30, 2004, each of which is incorporated herein by reference.

### BACKGROUND OF THE INVENTION

The invention relates to dosing regimens for the administration of combination therapies.

Combination therapy refers to the administration of two or more drugs for the treatment of a disease or disorder, or two or more comorbid conditions. While in some cases each component of the combination is acting independently of the other(s), in other cases the two drugs may be acting in a combinatorial manner, e.g., a synergistically, to produce a result that would not be achieved by the administration of the two drugs in a non-overlapping manner.

Notwithstanding the foregoing, it may be that combination therapy falling in the latter category may include drugs having different pharmacokinetic properties (e.g., different Tₘₐₓ times). In these cases, the full benefit of the combination therapy is not being realized.

Thus, there is a desire to develop better methods for combination therapy.

### SUMMARY OF THE INVENTION

In a first aspect, the invention features a method of enhancing the efficacy of a drug combination. The method includes the steps of i) administering a first drug in an amount sufficient to produce an effective plasma concentration of the first drug for a period of time T₁, and ii) administering a second drug in a manner sufficient to produce an effective plasma concentration of the second drug for at least 70% of time T₁. Desirably, the second drug is administered in a manner sufficient to produce an effective plasma concentration of the second drug for at least 75%, 80%, 85%, or even 90% of time T₁. Optionally, some or all of the second drug is formulated for sustained release, and/or the second drug is administered more than once during time T₁.

The invention also features a method of administering a drug combination. This method includes the steps of administering simultaneously, or within 30 minutes of one another, a first drug not formulated for sustained release and a second drug formulated for sustained release, wherein a) the first drug produces a peak plasma concentration at Tₘₐₓ₁, b) the second drug produces a peak plasma concentration at Tₘₐₓ₂, and c) Tₘₐₓ₂ is equal to or greater than Tₘₐₓ₁, provided that if the second drug were not formulated for sustained release Tₘₐₓ₁>Tₘₐₓ₂.

The invention further features a pharmaceutical composition including a unit dosage form including a first drug selected from tricyclic compounds, SSRIs, SNRIs, NsIDIs, antihistamines, and tetra-substituted pyrimidopyrimidines; and a second drug formulated for sustained release.

The invention features a kit including: (a) a first drug not formulated for sustained release, (b) a said second drug formulated for sustained release, and (c) instructions for administering simultaneously, or within 30 minutes of one another, said first drug and said second drug.

In the above methods, compositions, and kits, the first drug or second drug is desirably a tricyclic compound, SSRI, SNRI, NsIDI, antihistamine, corticosteroid, or a tetra-substituted pyrimidopyrimidine.

In any of the above methods, compositions, and kits, the first drug and the second drug are optionally formulated together in a unit dosage form. Unit dosage forms include, for example, a bilayer tablet having a first layer including the first drug not formulated for sustained release and a second layer including the second drug formulated for sustained release. The unit dosage form may also be a tablet having an inner core including the second drug formulated for sustained release and an outer coat including the first drug not formulated for sustained release. Furthermore, the unit dosage form may be a capsule having beads including the second drug formulated for sustained release and beads including the first drug not formulated for sustained release

Any of the unit dosage forms described herein may further include the second drug not formulated for sustained release.

In any of the above methods, compositions, and kits, the first drug may be a tricyclic compound and the second drug may be a corticosteroid, such as the combination of amoxapine and prednisolone or the combination of nortriptyline and budesonide; the first drug may be an SSRI and the second drug may be a corticosteroid, such as the combination of paroxetine and prednisolone; the first drug may be dipyridamole and the second drug may be a corticosteroid, such as prednisolone; the fist drug may be an NsIDI and the second drug may be an antihistamine, such as the combination of cyclosporin A and loratadine; or the first drug may be dipyridamole and the second drug may be an antihistamine, such as loratadine.

The compositions can be formulated for any route of administration. For example, the combination of nortriptyline and budesonide can be formulated for inhalation. Desirably, the combination is formulated for oral administration.

Compounds useful in the invention include those described herein in any of their pharmaceutically acceptable forms, including isomers such as diastereomers and enantiomers, salts, esters, solvates, and polymorphs thereof, as well as racemic mixtures and pure isomers of the compounds described herein.

The methods, compositions, and kits described herein can also be used to generate information useful, for example, for increasing investment in a company or increasing consumer demand for the methods, compositions, and/or kits.

The invention therefore features a method of increasing consumer demand for a pharmaceutical composition, therapeutic regimen, or kit described herein. The method includes the step of disseminating information about the pharmaceutical composition, therapeutic regimen, or kit.

The invention further features a method of increasing investment in a company seeking governmental approval for the sale of a pharmaceutical composition, therapeutic regimen, or kit described herein. The method includes the steps of i) disseminating information about the pharmaceutical composition, therapeutic regimen, or kit, and ii) disseminating information about the intent of the company to market the pharmaceutical composition, therapeutic regimen, or kit.

Consumer demand for a pharmaceutical composition or kit described herein, optionally with instructions to administer the pharmaceutical composition using a regimen described herein, can be increased by disseminating information about the utility, efficacy, or safety of the pharmaceutical composition, therapeutic regimen, or kit. Consumers include health maintenance organizations, hospitals, doctors, and patients. Typically, the information will be disseminated prior to a governmental approval for the sale of a composition, therapeutic regimen, or kit of the invention.

A company planning to sell a pharmaceutical composition or kit described herein, optionally with instructions to administer the pharmaceutical composition using a regimen described herein, can increase investment therein by disseminating information about the company's intention to seek governmental approval for the sale of and disseminating information about the pharmaceutical composition, therapeutic regimen, or kit. For example, the company can increase investment by disseminating information about in vivo studies conducted, or planned, by the company, including, without limitation, information about the toxicity, efficacy, or dosing requirements of a pharmaceutical composition, therapeutic regimen, or kit of the invention. The company can also increase investment by disseminating information about the projected date of governmental approval of a pharmaceutical composition, therapeutic regimen, or kit of the invention.

Information can be disseminated in any of a variety of ways, including, without limitation, by press release, public presentation (e.g., an oral or poster presentation at a trade show or convention), on-line posting at a web site, and mailing. Information about the pharmaceutical composition, therapeutic regimen, or kit can include, without limitation, a structure, diagram, figure, chemical name, common name, tradename, formula, reference label, or any other identifier that conveys the identity of the pharmaceutical composition, therapeutic regimen, or kit of the invention to a person.

By “in vivo studies” is meant any study in which a pharmaceutical composition, therapeutic regimen, or kit of the invention is administered to a mammal, including, without limitation, non-clinical studies, e.g., to collect data concerning toxicity and efficacy, and clinical studies.

By “projected date of governmental approval” is meant any estimate of the date on which a company will receive approval from a governmental agency to sell, e.g., to patients, doctors, or hospitals, a pharmaceutical composition, therapeutic regimen, or kit of the invention. A governmental approval includes, for example, the approval of a drug application by the Food and Drug Administration, among others.

By “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants. Exemplary SSRIs for use in the invention are described herein.

By “corticosteroid” is meant any naturally occurring or synthetic compound characterized by a hydrogenated cyclopentanoperhydrophenanthrene ring system and having immunosuppressive and/or antinflammatory activity. Naturally occurring corticosteriods are generally produced by the adrenal cortex. Synthetic corticosteriods may be halogenated. Examples corticosteroids are provided herein.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other drugs (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “treating” is meant administering or prescribing a pharmaceutical composition for the treatment or prevention of a disease or disorder.

By “patient” is meant any mammal (e.g., a human).

By “effective plasma concentration” is meant that the concentration of a drug in the plasma of a patient, in a combination of the invention, is in the range required to treat or prevent a disease or disorder in a clinically relevant manner. A sufficient amount of an active compound used to practice the present invention for therapeutic treatment of conditions caused by or contributing to, for example, an immunoinflammatory disease varies depending upon the manner of administration, the age, body weight, and general health of the patient. Ultimately, the prescribers will decide the appropriate amount and dosage regimen. Additionally, an effective amount may can be that amount of compound in the combination of the invention that is safe and efficacious in the treatment of a patient having a disease or disorder over each drug alone as determined and approved by a regulatory authority (such as the U.S. Food and Drug Administration).

By “enhances” or “enhancing” is meant that a treatment exhibits greater efficacy, or is less toxic, or safer in comparison to a treatment employing the same active ingredients, but not using the compositions or methods of the invention. Efficacy may be measured by a skilled practitioner using any standard method that is appropriate for a given indication.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hemolytic anemia; autoimmune hepatitis; Behcet's disease; Bell's palsy; bullous pemphigoid; cerebral ischaemia; cirrhosis; chronic obstructive pulmonary disease; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis /inflammation, psoriasis; psoriatic arthritis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-1 diabetes; ulcerative colitis; uveitis; vasculitis; and Wegener's granulomatosis.

“Non-dermal inflammatory disorders” include, for example, rheumatoid arthritis, inflammatory bowel disease, asthma, and chronic obstructive pulmonary disease.

“Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis.

As will be appreciated by one skilled in the art, a particular disease, disorder, or condition may be characterized as being both a proliferative skin disease and an inflammatory dermatosis. An example of such a disease is psorlasis.

By “sustained release” is meant a drug formulated for release at a controlled rate such that upon administration to a human, an effective plasma concentration of the drug is maintained for a period of time that is greater than 150%, 200%, 300%, 400%, or even 500% of the of time in which an effective plasma concentration is maintained upon administration of the same drug not formulated for sustained release, but otherwise administered under the same conditions.

By “not formulated for sustained release” is meant any formulation in which the removal of any one of the excipients present in the formulation fails to alter by more than 50% the length of time that an effective plasma concentration of the drug is maintained upon administration to a human.

By “Cₘₐₓ” is meant the maximum observed plasma concentration for an administered drug.

By “Tₘₐₓ” is meant the time at which Cₘₐₓ occurs following administration of a drug at time=0.

As used herein, “a period of time T₁” refers to the length of time over which a drug has an effective plasma concentration. Depending upon the amount administered, the bioavailability, and the elimination half-life, for the particular drug, time T₁ may be as little as 30 minutes or as long as 7 days. Typically, time T₁ will be between 30 minutes and 24 hours.

As used herein, “administered in a manner sufficient” refers to changes in either the amounts administered, dosing regimen, or formulation of a drug in order to more closely match the pharmacokinetic profile of another drug with which it is given.

The compositions and methods of the invention are useful for enhancing the efficacy of drug combinations in which the drugs of the combination have poorly overlapping pharmacokinetic profiles. The methods and compositions of the invention are designed to increase the length of time that each of the drugs administered for combination therapy is simultaneously present in the plasma of the subject in an amount that renders the two drugs together more therapeutically effective.

Other features and advantages of the invention will be apparent from the following detailed description, and from the claims.

### BRIEF DESCRIPTION OF THE DRAWINGS

FIG. 1 is a plot depicting the pharmacokinetic behavior of prednisolone and amoxapine administered orally to humans. The data show a poor match of pharmacokinetic curves for the two drugs.

FIG. 2 is a plot depicting the pharmacokinetic behavior of prednisolone and paroxetine administered orally to humans. The data show a poor match of pharmacokinetic curves for the two drugs.

### DETAILED DESCRIPTION

The invention provides methods, compositions, and kits for enhancing the efficacy of drug combinations. Administration of a drug combination in which one of the drugs is formulated for sustained release or administered repeatedly is useful where the pharmacokinetic profile of each drug must be modified to improve the efficacy of the combination. In the formulations of the invention, a pharmacokinetic profile is modified, for example, to increase the length of time that each of the drugs is simultaneously present in the plasma of the subject in an amount that renders the two drugs together more therapeutically effective than either drug administered alone. A sustained release formulation may be used to avoid frequent dosing that may be required in order to sustain the plasma levels of both drugs at a therapeutic level.

For example, a bilayer tablet can be formulated for an SSRI/steroid combination in which different custom granulations are made for each drug of the combination and the two drugs are compressed on a bi-layer press to form a single tablet. For example, 12.5 mg, 25 mg, 37.5 mg, or 50 mg of paroxetine, formulated for a sustained release that results in a paroxetine t_{1/2} of 15 to 20 hours may be combined in the same tablet with 3 mg of prednisolone, which is formulated such that the t_{1/2} approximates that of paroxetine. Examples of paroxetine extended-release formulations, including those used in bilayer tablets, can be found in U.S. Pat. No. 6,548,084. In addition to controlling the rate of predsnisolone release in vivo, an enteric or delayed release coat may be included that delays the start of drug release such that the Tₘₐₓ of predsnisolone approximates that of paroxetine (i.e. 5 to 10 hours).

The invention is described in greater detail below.

Combination Therapy

SSRI or SNRI in Combination with a Corticosteroid

A selective serotonin reuptake inhibitor (SSRI) or selective serotonin norepinephrine reuptake inhibitor (SNRI) can be administered in combination with a corticosteroid for the treatment of immunoinflammatory disorders as described in U.S. Ser. No. 10/670,488, entitled “Methods and Reagents for the Treatment of Diseases and Disorders Associated with increased Levels of Proinflammatory Cytokines,” filed Sep. 24, 2003. This application is incorporated herein by reference in its entirety.

NsIDI in Combination with an Antihistamine

A non-steroidal immunophilin-dependent immunosuppressant (NsIDI) can be administered in combination with an antihistamine for the treatment of immunoinflammatory disorders as described in U.S. Ser. No. 10/777,518, entitled “Combination Therapy for the Treatment of Immunoinflammatory Disorders,” filed Feb. 12, 2004. This application is incorporated herein by reference in its entirety.

Tricyclic Compound in Combination with a Corticosteroid

A tricyclic compound can be administered in combination with a corticosteroid for the treatment of immunoinflammatory disorders as described in Provisional Patent Application No. 60/520,446, entitled “Methods and Reagents for the Treatment of Diseases and Disorders Associated with Increased Levels of Proinflammatory Cytokines,” filed Nov. 13, 2003. This application is incorporated herein by reference in its entirety.

Dipyridamole in Combination with a Corticosteroid

Dipyridamole and other tetra-substituted pyrimidopyrimidines can be administered in combination with a corticosteroid for the treatment of immunoinflammatory disorders as described in U.S. Ser. No. 10/264,991, entitled “Combinations for the Treatment of Immunoinflammatory Disorders,” filed Oct. 4, 2002. This application is incorporated herein by reference in its entirety.

Dipyridamole in Combination with an Antihistamine

Dipyridamole and tetra-substituted pyrimidopyrimidines can be administered in combination with an antihistamine for the treatment of immunoinflammatory disorders as described in d described in Provisional Patent Application No. 60/512,415, entitled “Methods and Reagents for the Treatment of Diseases and Disorders Associated with Increased Levels of Proinflammatory Cytokines,” filed Oct. 15, 2003. This application is incorporated herein by reference in its entirety.

The Tₘₐₓ and elimination half-life data for a variety of drugs useful in the methods, compositions, and kits of the invention are provided in Table 1, below. With the exception of paroxetine/prednisolone and amoxapine/prednisolone, these data reflect the pharmacokinetic parameters for each drug administered as a monotherapy.

TABLE 1

| | | | Mean | Mean elimination |
|---|---|---|---|---|
| Drug Combinations | Route | Dose | Tₘₐₓ (Hours) | half life (Hours) |
| Loratadine (Claritin ®) | oral | 10 mg | 1.3 | 8.4 |
| Cyclosporine A (Sandimmune ®) | oral | 25 mg | 3.5 | 19 |
| Nortriptyline (Pamelor ®) | oral | 25 mg | 8 | 16 |
| Budesonide (Pulmicort Turbuhaler ®) | inhaled | 200 μg | 0.5 | ca. 2 |
| Dipyridamole (Persantine ®) | oral | 25 mg | 0.75 | 10 |
| Loratadine (Claritin ®) | oral | 10 mg | 1.3 | 8.4 |
| Paroxetine (Paxil ®)¹ | oral | 20 mg | 7 | 20.2 |
| Prednisolone¹ | oral | 3 mg | 1.9 | 2.7 |
| Dipyridamole (Persantine ®) | oral | 25 mg | 0.75 | 10 |
| Prednisolone | oral | 3 mg | 1.9 | 2.7 |
| Amoxapine (Asendin ®)² | oral | 100 mg | 2.4 | 9.7 |
| Prednisolone² | oral | 3 mg | 1.9 | 4.5 |
| Dipyridamole (Persantine ®) | oral | 25 mg | 0.75 | 10 |
| Prednisolone | oral | 3 mg | 1.9 | 2.7 |

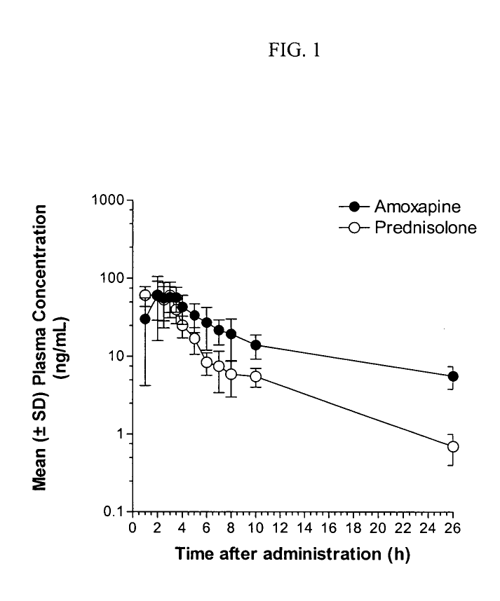

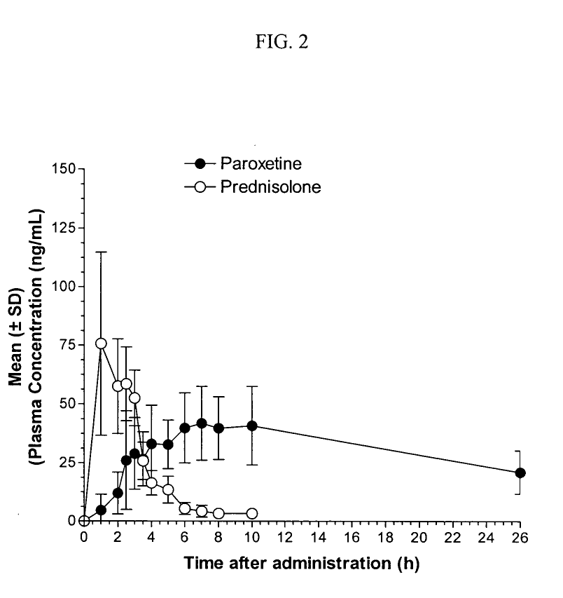

¹Data from PK study shown in FIG. 1.
²Data from PK study shown in FIG. 2.

SSRIs and SNRIs

The methods, compositions, and kits of the invention may employ an SSRI, or a structural or functional analog thereof. Suitable SSRIs include cericlamine (e.g., cericlamine hydrochloride); citalopram (e.g., citalopram hydrobromide); clovoxamine; cyanodothiepin; dapoxetine; escitalopram (escitalopram oxalate); femoxetine (e.g., femoxetine hydrochloride); fluoxetine (e.g., fluoxetine hydrochloride); fluvoxamine (e.g., fluvoxamine maleate); ifoxetine; indalpine (e.g., indalpine hydrochloride); indeloxazine (e.g., indeloxazine hydrochloride); litoxetine; milnacipran (e.g., minlacipran hydrochloride); paroxetine (e.g., paroxetine hydrochloride hemihydrate; paroxetine maleate; paroxetine mesylate); sertraline (e.g., sertraline hydrochloride); tametraline hydrochloride; viqualine; and zimeldine (e.g., zimeldine hydrochloride).

Functional analogs of SSRIs can also be used in the methods, compositions, and kits of the invention. Exemplary SSRI functional analogs are provided below. One class of SSRI analogs are SNRIs (selective serotonin norepinephrine reuptake inhibitors), which include venlafaxine, duloxetine, and 4-(2-fluorophenyl)-6-methyl-2-piperazinothieno[2,3-d]pyrimidine.

Standard recommended dosages for exemplary SSRIs are provided in Table 2, below. Other standard dosages are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002).

TABLE 2

| Compound | Standard Dose | |
|---|---|---|
| Fluoxetine | 20-80 | mg/day |
| Sertraline | 50-200 | mg/day |
| Paroxetine | 20-50 | mg/day |
| Fluvoxamine | 50-300 | mg/day |
| Citalopram | 10-80 | mg qid |
| Escitalopram | 10 | mg qid |

Generally, when administered orally to a human, the dosage of the SSRI is normally about 0.001 mg to 200 mg per day, desirably about 1 mg to 100 mg per day, and more desirably about 5 mg to 50 mg per day. Dosages up to 200 mg per day may be necessary. For administration of the SSRI by injection, the dosage is normally about 1 mg to 250 mg per day, desirably about 5 mg to 200 mg per day, and more desirably about 10 mg to 150 mg per day. Injections are desirably given one to four times daily. When systemically administered to a human, the dosage of the corticosteroid for use in combination with the SSRI is normally about 0.1 mg to 1500 mg per day, desirably about 0.5 mg to 10 mg per day, and more desirably about 0.5 mg to 5 mg per day.

Corticosteroids

The methods, compositions, and kits of the invention may employ a corticosteroid. Suitable corticosteroids include 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisoline 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclometasone dipropionate; aldosterone; algestone; alphaderm; amadinone; amcinonide; anagestone; androstenedione; anecortave acetate; beclomethasone; beclomethasone dipropionate; beclomethasone dipropionate monohydrate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; dihydroelatericin a; domoprednate; doxibetasol; ecdysone; ecdysterone; endrysone; enoxolone; flucinolone; fludrocortisone; fludrocortisone acetate; flugestone; flumethasone; flumethasone pivalate; flumoxonide; flunisolide; fluocinolone; fluocinolone acetonide; fluocinonide; 9-fluorocortisone; fluocortolone; fluorohydroxyandrostenedione; fluorometholone; fluorometholone acetate; fluoxymesterone; fluprednidene; fluprednisolone; flurandrenolide; fluticasone; fluticasone propionate; formebolone; formestane; formocortal; gestonorone; glyderinine; halcinonide; hyrcanoside; halometasone; halopredone; haloprogesterone; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; meclorisone; mecortolon; medrogestone; medroxyprogesterone; medrysone; megestrol; megestrol acetate; melengestrol; meprednisone; methandrostenolone; methylpredni solone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednisolamate; prednisolone; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone diacetate; triamcinolone hexacetonide; trimegestone; turkesterone; and wortmannin.

Standard recommended dosages for various steroid/disease combinations are provided in Table 3, below.

TABLE 3
Standard Recommended Corticosteroid Dosages

| Indication | Route | Drug | Dose | Schedule |
|---|---|---|---|---|
| Psoriasis | oral | prednisolone | 7.5-60 mg | per day or divided b.i.d. |
| | oral | prednisone | 7.5-60 mg | per day or divided b.i.d. |
| Asthma | inhaled | beclomethasone dipropionate | 42 μg/puff) | 4-8 puffs b.i.d. |
| | inhaled | budesonide | (200 μg/inhalation) | 1-2 inhalations b.i.d. |
| | inhaled | flunisolide | (250 μg/puff) | 2-4 puffs b.i.d. |
| | inhaled | fluticasone propionate | (44, 110 or 220 μg/puff) | 2-4 puffs b.i.d. |
| | inhaled | triamcinolone acetonide | (100 μg/puff) | 2-4 puffs b.i.d. |
| COPD | oral | prednisone | 30-40 mg | per day |
| Crohn's disease | oral | budesonide | 9 mg | per day |
| Ulcerative colitis | oral | prednisone | 40-60 mg | per day |
| | oral | hydrocortisone | 300 mg (IV) | per day |
| | oral | methylprednisolone | 40-60 mg | per day |
| Rheumatoid arthritis | oral | prednisone | 7.5-10 mg | per day |

Other standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

Steroid receptor modulators (e.g., antagonists and agonists) may be used as a substitute for or in addition to a corticosteroid in the methods, compositions, and kits of the invention. Thus, in one embodiment, the invention features the combination of an SSRI (or analog or metabolite thereof) and a glucocorticoid receptor modulator or other steroid receptor modulator, and methods of treating immunoinflammatory disorders therewith.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 20030176478, 20030171585, 20030120081, 20030073703, 2002015631, 20020147336, 20020107235, 20020103217, and 20010041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

NsIDIs

The methods, compositions, and kits of the invention may employ a non-steroidal immunophilin-dependent immunosuppressant (NsIDI).

NsIDls include calcineurin inhibitors (e.g., cyclosporines, tacrolimus, pimecrolimus), and rapamycin.

Cyclosporines

The cyclosporines are fungal metabolites that include a class of cyclic oligopeptides that act as immunosuppressants. Cyclosporine A, and its deuterated analogue ISAtx247, is a hydrophobic cyclic polypeptide consisting of eleven amino acids. Cyclosporine A binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T-cell-dependent immune response by inhibiting antigen-triggered signal transduction. This inhibition decreases the expression of proinflammatory cytokines, such as IL-2.

Many cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Publication No. 20020132763). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH—Cs (209-825), Allo-Thr-2-Cs, Norvaline-2-Cs, D-Ala (3-acetylamino)-8-Cs, Thr-2-Cs, and D-MeSer-3-Cs, D-Ser (O—CH₂CH₂—OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g., on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Cyclosporines can be administered either intravenously or orally, but oral administration is preferred. To counteract the hydrophobicity of cyclosporine A, an intravenous cyclosporine A is usually provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL™).

Typically, patient dosage of an oral cyclosporine varies according to the patient's condition, but some standard recommended dosages in prior art treatment regimens are provided herein. Patients undergoing organ transplant typically receive an initial dose of oral cyclosporine A in amounts between 12 and 15 mg/kg/day. Dosage is then gradually decreased by 5% per week until a 7-12 mg/kg/day maintenance dose is reached. For intravenous administration 2-6 mg/kg/day is preferred for most patients. For patients diagnosed as having Crohn's disease or ulcerative colitis, dosage amounts from 6-8 mg/kg/day are generally given. For patients diagnosed as having systemic lupus erythematosus, dosage amounts from 2.2-6.0 mg/kg/day are generally given. For psoriasis or rheumatoid arthritis, dosage amounts from 0.5-4 mg/kg/day are typical. Other useful dosages include 0.5-5 mg/kg/day, 5-10 mg/kg/day, 10-15 mg/kg/day, 15-20 mg/kg/day, or 20-25 mg/kg/day. Often cyclosporines are administered in combination with other immunosuppressive agents, such as glucocorticoids. Additional information is provided in Table 4.

TABLE 4
NsIDIs

| | Atopic | | | | | | |
|---|---|---|---|---|---|---|---|
| Compound | Dermatitis | Psoriasis | RA | Crohn's | UC | Transplant | SLE |
| CsA | N/A | 0.5-4 | 0.5-4 | 6-8 | 6-8 | ˜7-12 | 2.2-6.0 |
| (NEORAL) | | mg/kg/day | mg/kg/day | mg/kg/day | mg/kg/day | mg/kg/day | mg/kg/day |
| | | | | (oral- | (oral) | | |
| | | | | fistulizing) | | | |
| Tacrolimus | .03-0.1% | .05-1.15 | 1-3 | 0.1-0.2 | 0.1-0.2 | 0.1-0.2 | N/A |
| | cream/twice | mg/kg/day | mg/day | mg/kg/day | mg/kg/day | mg/kg/day | |
| | day (30 and | (oral) | (oral) | (oral) | (oral) | (oral) | |
| | 60 gram | | | | | | |
| | tubes) | | | | | | |
| Pimecrolimus | 1% | 40-60 | 40-60 | 80-160 | 160-240 | 40-120 | 40-120 |
| | cream/twice | mg/day | mg/day | mg/day | mg/day | mg/day | mg/day |
| | day (15, 30, | (oral) | (oral) | (oral) | (oral) | (oral) | (oral) |
| | 100 gram | | | | | | |
| | tubes) | | | | | | |

Legend
CsA = cyclosporine A
RA = rheumatoid arthritis
UC = ulcerative colitis
SLE = systemic lupus erythamatosus

Tacrolimus

Tacrolimus (PROGRAF, Fujisawa), also known as FK506, is an immunosuppressive drug that targets T-cell intracellular signal transduction pathways. Tacrolimus binds to an intracellular protein FK506 binding protein (FKBP-12) that is not structurally related to cyclophilin (Harding et al. Nature 341:758-7601, 1989; Siekienka et al. Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of NFAT, a nuclear component that initiates gene transcription required for lymphokine (e.g., IL-2, gamma interferon) production and T-cell activation. Thus, tacrolimus inhibits T-cell activation.

Tacrolimus is a macrolide antibiotic that is produced by Streptomyces tsukubaensis. It suppresses the immune system and prolongs the survival of transplanted organs. It is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 9% sodium chloride or 5% dextrose prior to injection. While oral administration is preferred, patients unable to take oral capsules may receive injectable tacrolimus. The initial dose should be administered no sooner than six hours after transplant by continuous intravenous infusion.

Tacrolimus and tacrolimus analogs are described by Tanaka et al., (J. Am. Chem. Soc., 109:5031, 1987), and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

While suggested dosages will vary with a patient's condition, standard recommended dosages and regimens are provided below. Patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Patients receiving oral tacrolimus capsules typically receive the first dose no sooner than six hours after transplant, or eight to twelve hours after intravenous tacrolimus infusion was discontinued. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus and Ascomycin Derivatives

Ascomycin is a close structural analog of FK506 and is a potent immunosuppressant. It binds to FKBP-12 and suppresses its proline rotamase activity. The ascomycin-FKBP complex inhibits calcineurin, a type 2B phosphatase.

Pimecrolimus (also known as SDZ ASM-981) is an 33-epi-chloro derivative of the ascomycin. It is produced by the strain Streptomyces hygroscopicus var. ascomyceitus. Like tacrolimus, pimecrolimus (ELIDEL™, Novartis) binds FKBP-12, inhibits calcineurin phosphatase activity, and inhibits T-cell activation by blocking the transcription of early cytokines. In particular, pimecrolimus inhibits IL-2 production and the release of other proinflammatory cytokines.

Pimecrolimus structural and functional analogs are described in U.S. Pat. No. 6,384,073. Pimecrolimus is particularly useful for the treatment of atopic dermatitis. Pimecrolimus is currently available as a 1% cream. While individual dosing will vary with the patient's condition, some standard recommended dosages are provided below. Oral pimecrolimus can be given for the treatment of psoriasis or rheumatoid arthritis in amounts of 40-60 mg/day. For the treatment of Crohn's disease or ulcerative colitis amounts of 80-160 mg/day pimecrolimus can be given. Patients having an organ transplant can be administered 160-240 mg/day of pimecrolimus. Patients diagnosed as having systemic lupus erythamatosus can be administered 40-120 mg/day of pimecrolimus. Other useful dosages of pimecrolimus include 0.5-5 mg/day, 5-10 mg/day, 10-30 mg/day, 40-80 mg/day, 80-120 mg/day, or even 120-200 mg/day.

Rapamycin

Rapamycin (Rapamune® sirolimus, Wyeth) is a cyclic lactone produced by Steptomyces hygroscopicus. Rapamycin is an immunosuppressive drug that inhibits T-lymphocyte activation and proliferation. Like cyclosporines, tacrolimus, and pimecrolimus, rapamycin forms a complex with the immunophilin FKBP-12, but the rapamycin-FKBP-12 complex does not inhibit calcineurin phosphatase activity. The rapamycin-immunophilin complex binds to and inhibits the mammalian target of rapamycin (mTOR), a kinase that is required for cell cycle progression. Inhibition of mTOR kinase activity blocks T-lymphocyte proliferation and lymphokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Everolimus (40-O-(2-hydroxyethyl)rapamycin; CERTICAN™; Novartis) is an immunosuppressive macrolide that is structurally related to rapamycin, and has been found to be particularly effective at preventing acute rejection of organ transplant when give in combination with cyclosporin A.

Rapamycin is currently available for oral administration in liquid and tablet formulations. RAPAMUNE™ liquid contains 1 mg/mL rapamycin that is diluted in water or orange juice prior to administration. Tablets containing 1 or 2 mg of rapamycin are also available. Rapamycin is preferably given once daily as soon as possible after transplantation. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1-mg/m²/day maintenance dose is used.

Tricyclic Compounds

The methods, compositions, and kits of the invention may employ a tricyclic compound. Tricyclic compounds include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. patent application Ser. Nos. 10/617,424 or 60/504,310.

Typically, patient dosages of maprotiline vary according to the patient's condition, but some standard recommended dosages are provided herein. Maprotiline, which is currently available in 25, 50, and 100 mg tablets, is most often administered in doses of 100-150 mg/day, although standard recommended dosages of 1-25 mg/day, 25-100 mg/day, 100-150 mg/day, 150-225 mg/day, or 225-350 mg/day can be administered. Most antidepressants are well absorbed when administered orally, although intramuscular administration of some TCAs (e.g., amitriptyline, clomipramine) is also possible.

Dipyridamole and Related Tetrasubstituted Pyrimidopyrimidines

The methods, compositions, and kits of the invention may employ dipyridamole or tetra-substituted pyrimidopyrimidines. Dipyridamole (2,6-bis(diethanolamino)-4,8-dipiperidinopyrimido(5,4-d)pyrimidine) is a tetra-substituted pyrimidopyrimidine that is used as a platelet inhibitor, e.g., to prevent blood clot formation following heart valve surgery and to reduced the moribundity associated with clotting disorders, including myocardial and cerebral infarction. Typically, anticoagulation therapy (prophylaxis or treatment) is effected by administering dipyridamole at about 75-200 mg b.i.d, t.i.d., or q.i.d. either alone or in combination with aspirin. In the invention, lower doses generally can be used, e.g., 20-80 mg, administered by any of the prior art routes.

Tetra-substituted pyrimidopyrimidines are structural analogs that can replace dipyridamole in the methods and compositions of this invention. Tetra-substituted pyrimidopyrimidines generally are of formula (I), described in U.S. Ser. No. 10/264,991 entitled “Combinations for the Treatment of Immunoinflammatory Disorders,” filed Oct. 4, 2002, and incorporated herein by reference in its entirety.

Exemplary tetra-substituted pyrimidopyrimidines that are useful in the methods and compositions of this invention include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine), mopidamole, dipyridamole monoacetate, NU3026 (2,6-di-(2,2-dimethyl-1,3-dioxolan-4-yl)-methoxy-4,8-di-piperidinopyrimidopyrimidine), NU3059 (2,6-bis-(2,3-dimethyoxypropoxy)-4,8-di-piperidinopyrimidopyrimidine), NU3060 (2,6-bis[N,N-di(2-methoxy)ethyl]-4,6-di-piperidinopyrimidopyrimidine), and NU3076 (2,6-bis(diethanolamino)-4,8-di-4-methoxybenzylaminopyrimidopyrimidine).

For oral, intramuscular, subcutaneous, topical, inhalation, rectal, vaginal and ophthalmic administration of the tetra-substituted pyrimidopyrimidine, the dosage used according to the invention is about 0.5-800 mg/day, preferably about 5-600 mg/day, 10-100 mg/day, and more preferably 0.5-50 mg/day. Administration can be one to four times daily for one day to one year, and may even be for the life of the patient. Chronic, long-term administration will be indicated in many cases. In some cases of serious illness, up to 1600 mg/day may be necessary. For intravenous administration of the tetra-substituted pyrimidopyrimidine, the dosage used is about 0.1-200 mg/day, preferably about 0.5-150 mg/day, 1-100 mg/day, and more preferably about 0.5-50 mg/day. Administration can be one to four times daily. Systemic dosing will result in steady-state plasma concentrations preferably of 0.1-7.0 μM, more preferably, 0.5-5.0 μM, and most preferably, 1.0-2.0 μM.

Antihistamines

The methods, compositions, and kits of the invention may employ an antihistamine. Antihistamines are compounds that block the action of histamine. Classes of antihistamines include:
(1) Ethanolamines (e.g., bromodiphenhydramine, carbinoxamine, clemastine, dimenhydrinate, diphenhydramine, diphenylpyraline, and doxylamine);
(2) Ethylenediamines (e.g., pheniramine, pyrilamine, tripelennamine, and triprolidine);
(3) Phenothiazines (e.g., diethazine, ethopropazine, methdilazine, promethazine, thiethylperazine, and trimeprazine);
(4) Alkylamines (e.g., acrivastine, brompheniramine, chlorpheniramine, desbrompheniramine, dexchlorpheniramine, pyrrobutamine, and triprolidine);
(5) Piperazines (e.g., buclizine, cetirizine, chlorcyclizine, cyclizine, meclizine, hydroxyzine);
(6) Piperidines (e.g., astemizole, azatadine, cyproheptadine, desloratadine, fexofenadine, loratadine, ketotifen, olopatadine, phenindamine, and terfenadine);
(7) Atypical antihistamines (e.g., azelastine, levocabastine, methapyrilene, and phenyltoxamine).

In the methods, compositions, and kits of the invention, both non-sedating and sedating antihistamines may be employed. Particularly desirable antihistamines for use in the methods, compositions, and kits of the invention are non-sedating antihistamines such as loratadine and desloratadine. Sedating antihistamines can also be used in the methods, compositions, and kits of the invention. Preferred sedating antihistamines for use in the methods, compositions, and kits of the invention are azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the methods and compositions of the invention are acrivastine; ahistan; antazoline; astemizole; azelastine (e.g., azelsatine hydrochloride); bamipine; bepotastine; bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine difumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Structural analogs of antihistamines may also be used in according to the invention. Antihistamine analogs include, without limitation, 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane. Other compounds that are suitable for use in the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060. Still other compounds that are suitable for use in the invention are described in U.S. Pat. Nos. 3,956,296; 4,254,129; 4,254,130; 4,282,833; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

Standard recommended dosages for several exemplary antihistamines are shown in Table 5. Other standard dosages are provided, e.g., in the Merck Manual of Diagnosis & Therapy (17th Ed. M H Beers et al., Merck & Co.) and Physicians' Desk Reference 2003 (57th Ed. Medical Economics Staff et al., Medical Economics Co., 2002).

TABLE 5

| Compound | Standard Dose |
|---|---|
| Desloratadine | 5 mg/once daily |
| Thiethylperazine | 10 mg/1-3 times daily |
| Bromodiphenhydramine | 12.5-25 mg/every 4-6 |
| | hours |
| Promethazine | 25 mg/twice daily |
| Cyproheptadine | 12-16 mg/day |
| Loratadine | 10 mg/once daily |
| Clemizole | 100 mg given as IV or IM |
| Azatadine | 1-2 mg/twice daily |
| Cetirizine | 5-10 mg/once daily |
| Chlorpheniramine | 2 mg/every 6 hours or |
| | 4 mg/every 6 hours |
| Dimenhydramine | 50-100 mg/every 4-6 hours |
| Diphenydramine | 25 mg/every 4-6 hours or |
| | 38 mg/every 4-6 hours* |
| Doxylamine | 25 mg/once daily or 12.5 mg/ |
| | every four hours* |
| Fexofenadine | 60 mg/twice daily or 180 mg/ |
| | once daily |
| Meclizine | 25-100 mg/day |
| Pyrilamine | 30 mg/every 6 hours |
| Tripelennamine | 25-50 mg/every 4 to 6 |
| | hours or 100 mg/twice |
| | daily (extended release)* |

Loratadine (CLARITIN®) is a tricyclic piperidine that acts as a selective peripheral histamine H1-receptor antagonist. We report herein that loratadine and structural and functional analogs thereof, such as piperidines, tricyclic piperidines, histamine H1-receptor antagonists, are useful in the anti-immunoinflammatory combination of the invention for the treatment of immunoinflammatory disorders, transplanted organ rejection, and graft versus host disease.

Loratadine functional and/or structural analogs include other H1-receptor antagonists, such as AHR-11325, acrivastine, antazoline, astemizole, azatadine, azelastine, bromopheniramine, carebastine, cetirizine, chlorpheniramine, chlorcyclizine, clemastine, cyproheptadine, descarboethoxyloratadine, dexchlorpheniramine, dinienhydrinate, diphenylpyraline, diphenhydramine, ebastine, fexofenadine, hydroxyzine ketotifen, lodoxamide, levocabastine, methdilazine, mequitazine, oxatomide, pheniramine pyrilamine, promethazine, pyrilamine, setastine, tazifylline, temelastine, terfenadine, trimeprazine, tripelennamine, triprolidine, utrizine, and similar compounds (described, e.g., in U.S. Pat. Nos. 3,956,296, 4,254,129, 4,254,130, 4,283,408, 4,362,736, 4,394,508, 4,285,957, 4,285,958, 4,440,933, 4,510,309, 4,550,116, 4,692,456, 4,742,175, 4,908,372, 5,204,249, 5,375,693, 5,578,610, 5,581,011, 5,589,487, 5,663,412, 5,994,549, 6,201,124, and 6,458,958).

Loratadine, cetirizine, and fexofenadine are second-generation H1-receptor antagonists that lack the sedating effects of many first generation H1-receptor antagonists. Piperidine H1-receptor antagonists include loratadine, cyproheptadine hydrochloride (PERIACTIN), and phenindiamine tartrate (NOLAHIST). Piperazine H1-receptor antagonists include hydroxyzine hydrochloride (ATARAX), hydroxyzine pamoate (VISTARIL), cyclizine hydrochloride (MAREZINE), cyclizine lactate, and meclizine hydrochloride.

Loratadine oral formulations include tablets, redi-tabs, and syrup. Loratadine tablets contain 10 mg micronized loratadine. Loratadine syrup contains 1 mg/ml micronized loratadine, and reditabs (rapidly-disintegrating tablets) contain 10 mg micronized loratadine in tablets that disintegrate quickly in the mouth. While suggested dosages will vary with a patient's condition, standard recommended dosages are provided below. Loratadine is typically administered once daily in a 10 mg dose, although other daily dosages useful in the anti-immunoinflammatory combination of the invention include 0.01-0.05 mg, 0.05-1 mg, 1-3 mg, 3-5 mg, 5-10 mg, 10-15 mg, 15-20 mg, 20-30 mg, and 30-40 mg.

Loratadine is rapidly absorbed following oral administration. It is metabolized in the liver to descarboethoxyloratadine by cytochrome P450 3A4 and cytochrome P450 2D6. Loratadine metabolites are also useful in the anti-immunoinflammatory combination of the invention.

Administration

Using the methods of the invention, the drugs are administered within 30 minutes of each other, or simultaneously. The drugs may be formulated together as a single composition, or may be formulated and administered separately. It may be desirable to administer to the patient other compounds, such as an NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies of the invention are especially useful for the treatment of immunoinflammatory disorders in combination with other anti-cytokine agents or agents that modulate the immune response to positively effect disease, such as agents that influence cell adhesion, or biologics (i.e., agents that block the action of IL-6, IL-1, IL-2, IL-12, IL-15 or TNFα (e.g., etanercept, adelimumab, infliximab, or CDP-870). In this example (that of agents blocking the effect of TNFα), the combination therapy reduces the production of cytokines, etanercept or infliximab act on the remaining fraction of inflammatory cytokines, providing enhanced treatment.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment. Additionally, a person having a greater risk of developing an inflammatory disease (e.g., a person who is undergoing age-related hormonal changes) may receive treatment to inhibit or delay the onset of symptoms.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration.

In combination therapy, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

Formulation of Pharmaceutical Compositions

The administration of a combination of the invention may be by any suitable means that results in the desired therapeutic outcome. A component, or the entire combination, may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, the first and second drugs may be formulated together or separately. Desirably, the first and second drugs are formulated together for the simultaneous or near simultaneous administration of the drugs. Such co-formulated compositions can include, for example, the SSRI and the steroid formulated together in the same pill, capsule, liquid, etc. By using different formulation strategies for different drugs, the pharmacokinetic profiles for each drug can be suitably matched.

The individually or separately formulated drugs can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Sustained Release Formulations

Administration of any combination of the invention in which one of the active agents is formulated for sustained release is useful where one of the agents has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; (iii) a short biological half-life; or (iv) the pharmacokinetic profile of each component must be modified to improve the efficacy of the combination. In the formulations of the invention, a pharmacokinetic profile can be modified, for example, to increase the length of time that each of the agents is simultaneously present in the plasma of the subject in an amount that renders the two agents together more therapeutically effective than either agent administered alone. Accordingly, a sustained release formulation of one of the agents may be used to avoid frequent dosing that may be required in order to sustain the plasma levels of both agents at a therapeutic level. For example, in preferable oral pharmaceutical compositions of the invention, half-life and mean residency times from 10 to 20 hours for one or both agents of the combination of the invention are observed.

Many strategies can be pursued to obtain sustained release in which the rate of release outweighs the rate of metabolism of the therapeutic compound. For example, sustained release can be obtained by the appropriate selection of formulation parameters and ingredients (e.g., appropriate sustained release compositions and coatings). Examples include single or multiple unit tablet or capsule compositions, oil solutions, suspensions, emulsions, microcapsules, microspheres, nanoparticles, patches, and liposomes. The release mechanism can be controlled such that the a drug of the combination is released at period intervals, the release could be simultaneous, or a delayed release of one of the agents of the combination can be affected, when the early release of one particular agent is preferred over the other.

Sustained release formulations may include a degradable or nondegradable polymer, hydrogel, organogel, or other physical construct that modifies the bioabsorption, half-life or biodegradation of the agent. The sustained release formulation can be a material that is painted or otherwise applied onto the afflicted site, either internally or externally. In one example, the invention provides a biodegradable bolus or implant that is surgically inserted at or near a site of interest (for example, proximal to an arthritic joint). In another example, the sustained release formulation implant can be inserted into an organ, such as in the lower intestine for the treatment inflammatory bowel disease.

Hydrogels can be used in sustained release formulations for the combinations of the present invention. Such polymers include those described in U.S. Pat. No. 5,626,863. For example, hydrogels be gelled into a biodegradable network that can be used to entrap and homogeneously disperse combinations of the invention for delivery at a controlled rate.

Chitosan and mixtures of chitosan with carboxymethylcellulose sodium (CMC—Na) have been used as vehicles for the sustained release of drugs, as described by Inouye et al., Drug Design and Delivery 1: 297-305, 1987. Mixtures of these compounds and agents of the combinations of the invention, when compressed under 200 kg/cm², form a tablet from which the active agent is slowly released upon administration to a subject. The release profile can be changed by varying the ratios of chitosan, CMC—Na, and active agent(s). The tablets can also contain other additives, including lactose, CaHPO₄ dihydrate, sucrose, crystalline cellulose, or croscarmellose sodium. Several examples are given in Table 6.

TABLE 6

| Materials | Tablet components (mg) | | | | | | | | | | | |
|---|---|---|---|---|---|---|---|---|---|---|---|---|
| Active agent | 20 | 20 | 20 | 20 | 20 | 20 | 20 | 20 | 20 | 20 | 20 | 20 |
| Chitosan | 10 | 10 | 10 | 10 | 10 | 20 | 3.3 | 20 | 3.3 | 70 | 40 | 28 |
| Lactose | | 110 | | | | 220 | 36.7 | | | | | |
| CMC-Na | 60 | 60 | 60 | 60 | 60 | 120 | 20 | 120 | 20 | | 30 | 42 |
| CaHPO₄*2H₂O | | | 110 | | | | | 220 | 36.7 | 110 | 110 | 110 |
| Sucrose | 110 | | | | | | | | | | | |
| Crystalline | | | | | 110 | | | | | | | |
| Cellulose | | | | | | | | | | | | |
| Croscarmellose Na | | | | 110 | | | | | | | | |

Baichwal, in U.S. Pat. No. 6,245,356, describes a sustained release oral solid dosage forms that includes agglomerated particles of a therapeutically active medicament (e.g., a combination or component thereof of the present invention) in amorphous form, a gelling agent, an ionizable gel strength enhancing agent and an inert diluent. The gelling agent can be a mixture of a xanthan gum and a locust bean gum capable of cross-linking with the xanthan gum when the gums are exposed to an environmental fluid. Preferably, the ionizable gel enhancing agent acts to enhance the strength of cross-linking between the xanthan gum and the locust bean gum and thereby prolonging the release of the medicament component of the formulation. In addition to xanthan gum and locust bean gum, acceptable gelling agents that may also be used include those gelling agents well-known in the art. Examples include naturally occurring or modified naturally occurring gums such as alginates, carrageenan, pectin, guar gum, modified starch, hydroxypropylmethylcellulose, methylcellulose, and other cellulosic materials or polymers, such as, for example, sodium carboxymethylcellulose and hydroxypropyl cellulose, and mixtures of the foregoing.

In another formulation useful for the combinations of the invention, Baichwal and Staniforth in U.S. Pat. No. 5,135,757 describe a free-flowing slow release granulation for use as a pharmaceutical excipient that includes from about 20 to about 70 percent or more by weight of a hydrophilic material that includes a heteropolysaccharide (such as, for example, xanthan gum or a derivative thereof) and a polysaccharide material capable of cross-linking the heteropolysaccharide (such as, for example, galactomannans, and most preferably locust bean gum) in the presence of aqueous solutions, and from about 30 to about 80 percent by weight of an inert pharmaceutical filler (such as, for example, lactose, dextrose, sucrose, sorbitol, xylitol, fructose or mixtures thereof). After mixing the excipient with a combination, or combination agent, of the invention, the mixture is directly compressed into solid dosage forms such as tablets. The tablets thus formed slowly release the medicament when ingested and exposed to gastric fluids. By varying the amount of excipient relative to the medicament, a slow release profile can be attained.

Combinations of the invention can be formulated as provided in U.S. Pat. No. 5,007,790, which describes sustained release oral drug-dosage forms that release a drug in solution at a rate controlled by the solubility of the drug. The dosage form includes a tablet or capsule that includes a plurality of particles of a dispersion of a limited solubility drug in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles, assuring that drug reaches the stomach in the solution state which is less injurious to the stomach than solid-state drug. The programmed eventual dissolution of the polymer depends upon the nature of the polymer and the degree of crosslinking. The polymer is nonfibrillar and substantially water soluble in its uncrosslinked state, and the degree of crosslinking is sufficient to enable the polymer to remain insoluble for the desired time period, normally at least from about 4 hours to 8 hours up to 12 hours, with the choice depending upon the drug incorporated and the medical treatment involved. Examples of suitable crosslinked polymers that may be used in the invention are gelatin, albumin, sodium alginate, carboxymethyl cellulose, polyvinyl alcohol, and chitin. Depending upon the polymer, crosslinking may be achieved by thermal or radiation treatment or through the use of crosslinking agents such as aldehydes, polyamino acids, metal ions and the like.

Silicone microspheres for pH-controlled gastrointestinal drug delivery that are useful in the formulation of the combinations of the invention have been described by Carelli et al., Int. J. Pharmaceutics 179: 73-83, 1999. The microspheres so described are pH-sensitive semi-interpenetrating polymer hydrogels made of varying proportions of poly(methacrylic acid-co-methylmethacrylate) (Eudragit L100 or Eudragit S100) and crosslinked polyethylene glycol 8000 that are encapsulated into silicone microspheres in the 500 to 1000 μm size range.

Slow-release formulations can include a coating which is not readily water-soluble but which is slowly attacked and removed by water, or through which water can slowly permeate. Thus, combinations of the invention can be spray-coated with a solution of a binder under continuously fluidizing conditions, such as describe by Kitamori et al., U.S. Pat. No. 4,036,948. Examples of water-soluble binders include pregelatinized starch (e.g., pregelatinized corn starch, pregelatinized white potato starch), pregelatinized modified starch, water-soluble celluloses (e.g. hydroxypropyl-cellulose, hydroxymethyl-cellulose, hydroxypropylmethyl-cellulose, carboxymethyl-cellulose), polyvinylpyrrolidone, polyvinyl alcohol, dextrin, gum arabicum and gelatin, organic solvent-soluble binders, such as cellulose derivatives (e.g., cellulose acetate phthalate, hydroxypropylmethyl-cellulose phthalate, ethylcellulose).

Combinations of the invention, or a component thereof, with sustained release properties can also be formulated by spray drying techniques. In one example, as described by Espositio et al., Pharm. Dev. Technol. 5: 267-78, 2000, prednisolone was encapsulated in methyacrylate microparticles (Eudragit RS) using a Mini Spray Dryer, model 190 (Buchi, Laboratorium Technik AG, Flawil, Germany). Optimal conditions for microparticle formation were found to be a feed (pump) rate of 0.5 mL/min of a solution containing 50 mg prednisolone in 10 mL of acetonitrile, a flow rate of nebulized air of 600 L/hr, dry air temperature heating at 80° C., and a flow rate of aspirated drying air of 28 m³/hr.

Yet another form of sustained release combinations can be prepared by microencapsulation of combination agent particles in membranes which act as microdialysis cells. In such a formulation, gastric fluid permeates the microcapsule walls and swells the microcapsule, allowing the active agent(s) to dialyze out (see, for example, Tsuei et al., U.S. Pat. No. 5,589,194). One commercially available sustained release system of this kind consists of microcapsules having membranes of acacia gum/gelatine/ethyl alcohol. This product is available from Eurand Limited (France) under the trade name Diffucaps™. Microcapsules so formulated might be carried in a conventional gelatine capsule or tabletted.

Extended- and/or sustained release formulations of both SSRIs and corticosteroids are known. For example, Paxil CR®, commercially available from GlaxoSmithKline, is an extended release form of paroxetine hydrochloride in a degradable polymeric matrix (GEOMATRIX™, see also U.S. Pat. Nos. 4,839,177, 5,102,666, and 5,422,123), which also has an enteric coat to delay the start of drug release until after the tablets have passed through the stomach. For example, U.S. Pat. No. 5,102,666 describes a polymeric sustained release composition including a reaction complex formed by the interaction of (1) a calcium polycarbophil component which is a water-swellable, but water insoluble, fibrous cross-linked carboxy-functional polymer, the polymer containing (a) a plurality of repeating units of which at least about 80% contain at least one carboxyl functionality, and (b) about 0.05 to about 1.5% cross-linking agent substantially free from polyalkenyl polyether, the percentages being based upon the weights of unpolymerised repeating unit and cross-linking agent, respectively, with (2) water, in the presence of an active agent selected from the group consisting of SSRIs such as paroxetine. The amount of calcium polycarbophil present is from about 0.1 to about 99% by weight, for example about 10%. The amount of active agent present is from about 0.0001 to about 65% by weight, for example between about 5 and 20%. The amount of water present is from about 5 to about 200% by weight, for example between about 5 and 10%. The interaction is carried out at a pH of between about 3 and about 10, for example about 6 to 7. The calcium polycarbophil is originally present in the form of a calcium salt containing from about 5 to about 25% calcium.

Other extended-release formulation examples are described in U.S. Pat. No. 5,422,123. This formulation includes (a) a deposit-core having an effective amount of the active substance and having defined geometric form, and (b) a support-plafform applied to the deposit-core, wherein the deposit-core contains at least the active substance, and at least one member selected from the group consisting of (1) a polymeric material which swells on contact with water or aqueous liquids and a gellable polymeric material wherein the ratio of the swellable polymeric material to the gellable polymeric material is in the range 1:9 to 9:1, and (2) a single polymeric material having both swelling and gelling properties, and wherein the support-platform is an elastic support, applied to the deposit-core so that it partially covers the surface of the deposit-core and follows changes due to hydration of the deposit-core and is slowly soluble and/or slowly gellable in aqueous fluids. The support-platform may include polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support-platform, for example about 15 to 20%. Binder(s), hydrophilic agent(s) and hydrophobic agent(s) typically total up to about 50% by weight of the support-platform, for example about 40 to 50%.

In another example, an extended-release formulation for venlafaxine (Effexor XR®) is commercially available from Wyeth Pharmaceuticals. This formulation includes venlafaxine hydrochloride, microcrystalline cellulose and hydroxypropylmethylcellulose, coated with a mixture of ethyl cellulose and hydroxypropylmethylcellulose (see U.S. Pat. Nos. 6,403,120 and 6,419,958).

A sustained release formulation of budesonide (3 mg capsules) for the treatment of inflammatory bowel disease is available from AstraZeneca (sold as “Entocort™”). A sustained release formulation useful for corticosteroids is also described in U.S. Pat. No. 5,792,476, where the formulation includes 2.5-7 mg of a glucocorticoid as active substance with a regulated sustained release such that at least 90% by weight of the glucocorticoid is released during a period of about 40-80 min, starting about 1-3 h after the entry of the glucocorticoid into the small intestine of the patient. To make these low dose levels of active substance possible, the active substance, i.e., the glucocorticoid, such as prednisolone or prednisone, is micronised, suitably mixed with known diluents, such as starch and lactose, and granulated with PVP (polyvinylpyrrolidone). Further, the granulate is laminated with a sustained release inner layer resistant to a pH of 6.8 and a sustained release outer layer resistant to a pH of 1.0. The inner layer is made of Eudragit®RL (copolymer of acrylic and methacrylic esters with a low content of quaternary ammonium groups) and the outer layer is made of Eudragit®L (anionic polymer synthesized from methacrylic acid and methacrylic acid methyl ester).

A bilayer tablet can be formulated for any combination of the invention in which different custom granulations are made for each agent of the combination and the two agents are compressed on a bi-layer press to form a single tablet. For example, 12.5 mg, 25 mg, 37.5 mg, or 50 mg of paroxetine, formulated for a sustained release that results in a paroxetine t_{1/2} of 15 to 20 hours may be combined in the same tablet with 3 mg of prednisolone, which is formulated such that the t/₂ approximates that of paroxetine. Examples of paroxetine extended-release formulations, including those used in bilayer tablets, can be found in U.S. Pat. No. 6,548,084. In addition to controlling the rate of predsnisolone release in vivo, an enteric or delayed release coat may be included that delays the start of drug release such that the Tₘₐₓ of predsnisolone approximate that of paroxetine (i.e. 5 to 10 hours).

Cyclodextrins are cyclic polysaccharides containing naturally occurring D(+)-glucopyranose units in an α-(1,4) linkage. Alpha-, beta- and gamma-cyclodextrins, which contain, respectively, six, seven or eight glucopyranose units, are most commonly used and suitable examples are described in WO91/11172, WO94/02518 and WO98/55148. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. The side on which the secondary hydroxyl groups are located has a wider diameter than the side on which the primary hydroxyl groups are located. The hydrophobic nature of the cyclodextrin inner cavity allows for the inclusion of a variety of compounds. (Comprehensive Supramolecular Chemistry, Volume 3, J. L. Atwood et al., eds., Pergamon Press (1996); Cserhati, Analytical Biochemistry 225: 328-32, 1995; Husain et al., Applied Spectroscopy 46: 652-8, 1992. Cyclodextrins have been used as a delivery vehicle of various therapeutic compounds by forming inclusion complexes with various drugs that can fit into the hydrophobic cavity of the cyclodextrin or by forming non-covalent association complexes with other biologically active molecules. U.S. Pat. No. 4,727,064 describes pharmaceutical preparations consisting of a drug with substantially low water solubility and an amorphous, water-soluble cyclodextrin-based mixture in which the drug forms an inclusion complex with the cyclodextrins of the mixture.

Formation of a drug-cyclodextrin complex can modify the drug's solubility, dissolution rate, bioavailability, and/or stability properties. For example, cyclodextrins have been described for improving the bioavailability of prednisolone, as described by Uekama et al., J. Pharm Dyn. 6: 124-7, 1983. A β-cyclodextrin/prednisolone complex can be prepared by adding both components to water and stirring at 25° C. for 7 days. The resultant precipitate recovered is a 1:2 prednisolone/cyclodextrin complex.

Sulfobutylether-β-cyclodextrin (SBE-β-CD, commercially available from CyDex, Inc, Overland Park, Kans., USA and sold as CAPTISOL®) can also be used as an aid in the preparation of sustained release formulations of agents of the combinations of the present invention. For example, a sustained release tablet has been prepared that includes prednisolone and SBE-β-CD compressed in a hydroxypropyl methylcellulose matrix (see Rao et al., J. Pharm. Sci. 90: 807-16, 2001). In another example of the use of various cyclodextrins, EP 1109806 B 1 describes cyclodextrin complexes of paroxetine, where α-, γ-, or β-cyclodextrins [including eptakis(2-6-di-O-methyl)-β-cyclodextrin, (2,3,6-tri-O-methyl)-β-cyclodextrin, monosuccinyl eptakis(2,6-di-O-methyl)-β-cyclodextrin, or 2-hydroxypropyl-β-cyclodextrin] in anhydrous or hydrated form formed complex ratios of agent to cyclodextrin of from 1:0.25 to 1:20 can be obtained.

Polymeric cyclodextrins have also been prepared, as described in U.S. patent application Ser. Nos. 10/021,294 and 10/021,312. The cyclodextrin polymers so formed can be useful for formulating agents of the combinations of the present invention. These multifunctional polymeric cyclodextrins are commercially available from Insert Therapeutics, Inc., Pasadena, Calif., USA.

As an alternative to direct complexation with agents, cyclodextrins may be used as an auxiliary additive, e.g. as a carrier, diluent or solubiliser. Formulations that include cyclodextrins and other agents of the combinations of the present invention (e.g., tricyclic compounds, SSRIs, SNRIs, NsIDIs, antihistamines, corticosteroids, and/or a tetra-substituted pyrimidopyrimidines) can be prepared by methods similar to the preparations of the cyclodextrin formulations described herein.

Liposomal Formulations

One or both components of the combinations of the invention, or mixtures of the two components together, can be incorporated into liposomal carriers for administration. The liposomal carriers are composed of three general types of vesicle-forming lipid components. The first includes vesicle-forming lipids which will form the bulk of the vesicle structure in the liposome. Generally, these vesicle-forming lipids include any amphipathic lipids having hydrophobic and polar head group moieties, and which (a) can form spontaneously into bilayer vesicles in water, as exemplified by phospholipids, or (b) are stably incorporated into lipid bilayers, with its hydrophobic moiety in contact with the interior, hydrophobic region of the bilayer membrane, and its polar head group moiety oriented toward the exterior, polar surface of the membrane.

The vesicle-forming lipids of this type are preferably ones having two hydrocarbon chains, typically acyl chains, and a polar head group. Included in this class are the phospholipids, such as phosphatidylcholine (PC), PE, phosphatidic acid (PA), phosphatidylinositol (PI), and sphingomyelin (SM), where the two hydrocarbon chains are typically between about 14-22 carbon atoms in length, and have varying degrees of unsaturation. The above-described lipids and phospholipids whose acyl chains have a variety of degrees of saturation can be obtained commercially, or prepared according to published methods. Other lipids that can be included in the invention are glycolipids and sterols, such as cholesterol.

The second general component includes a vesicle-forming lipid which is derivatized with a polymer chain which will form the polymer layer in the composition. The vesicle-forming lipids which can be used as the second general vesicle-forming lipid component are any of those described for the first general vesicle-forming lipid component. Vesicle forming lipids with diacyl chains, such as phospholipids, are preferred. One exemplary phospholipid is phosphatidylethanolamine (PE), which provides a reactive amino group which is convenient for coupling to the activated polymers. An exemplary PE is distearyl PE (DSPE).

The preferred polymer in the derivatized lipid, is polyethyleneglycol (PEG), preferably a PEG chain having a molecular weight between 1,000-15,000 daltons, more preferably between 2,000 and 10,000 daltons, most preferably between 2,000 and 5,000 daltons. Other hydrophilic polymers which may be suitable include polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose.

Additionally, block copolymers or random copolymers of these polymers, particularly including PEG segments, may be suitable. Methods for preparing lipids derivatized with hydrophilic polymers, such as PEG, are well known e.g., as described in U.S. Pat. No. 5,013,556.

A third general vesicle-forming lipid component, which is optional, is a lipid anchor by which a targeting moiety is anchored to the liposome, through a polymer chain in the anchor. Additionally, the targeting group is positioned at the distal end of the polymer chain in such a way so that the biological activity of the targeting moiety is not lost. The lipid anchor has a hydrophobic moiety which serves to anchor the lipid in the outer layer of the liposome bilayer surface, a polar head group to which the interior end of the polymer is covalently attached, and a free (exterior) polymer end which is or can be activated for covalent coupling to the targeting moiety. Methods for preparing lipid anchor molecules of this types are described below.

The lipids components used in forming the liposomes are preferably present in a molar ratio of about 70-90 percent vesicle forming lipids, 1-25 percent polymer derivatized lipid, and 0.1-5 percent lipid anchor. One exemplary formulation includes 50-70 mole percent underivatized PE, 20-40 mole percent cholesterol, 0.1-1 mole percent of a PE-PEG (3500) polymer with a chemically reactive group at its free end for coupling to a targeting moiety, 5-10 mole percent PE derivatized with PEG 3500 polymer chains, and 1 mole percent alpha-tocopherol.

The liposomes are preferably prepared to have substantially homogeneous sizes in a selected size range, typically between about 0.03 to 0.5 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less.

Other established liposomal formulation techniques can be applied as needed. For example, the use of liposomes to facilitate cellular uptake is described in U.S. Pat. Nos. 4,897,355 and 4,394,448.

Administration of each drug in any of the combinations described herein can, independently, be one to four times daily for one day to one year, and may even be for the life of the patient. Chronic, long-term administration will be indicated in many cases.

### OTHER EMBODIMENTS

All publications, patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

1. A method of enhancing the efficacy of a combination of a first and second drug, said method comprising:
i) administering said first drug to a patient in an amount sufficient to produce an effective plasma concentration for a period of time T₁, and
ii) administering said second drug to said patient in a manner sufficient to produce an effective plasma concentration for at least 70% of time T₁.

2. The method of claim 1, wherein some or all of said second drug is formulated for sustained release.

3. The method of claim 1, wherein said second drug is administered more than once during said time T₁.

4. The method of claim 1, wherein said second drug is administered in a manner sufficient to produce an effective plasma concentration of said second drug for at least 80% of time T₁.

5. A method of administering a combination of a first and second drug to a patient, said method comprising administering simultaneously, or within 30 minutes of one another, said first drug not formulated for sustained release and said second drug formulated for sustained release,
wherein:
a) said first drug produces a peak plasma concentration at Tₘₐₓ₁,
b) said second drug produces a peak plasma concentration at Tₘₐₓ₂, and
c) Tₘₐₓ₂ is equal to or greater than Tₘₐₓ₁,
provided that if said second drug were not formulated for sustained release Tₘₐₓ₁>Tₘₐₓ₂.

6. The method of claim 1 or 5, wherein said first drug and said second drug are formulated together in a unit dosage form.

7. The method of claim 6 wherein said unit dosage form is a bilayer tablet having a first layer comprising said first drug not formulated for sustained release and a second layer comprising said second drug formulated for sustained release.

8. The method of claim 6 wherein said unit dosage form is a tablet having an inner core comprising said second drug formulated for sustained release and an outer coat comprising said first drug not formulated for sustained release.

9. The method of claim 6, wherein said unit dosage form is a capsule having beads comprising said second drug formulated for sustained release and beads comprising said first drug not formulated for sustained release.

10. The method of claim 9, wherein said capsule further comprises beads comprising said second agent not formulated for sustained release.

11. The method of claim 1 or 5, wherein said first drug or said second drug is a tricyclic compound, SSRI, SNRI, NsIDI, antihistamine, corticosteroid, or tetra-substituted pyrimidopyrimidine.

12. The method of claim 11, wherein said second drug is a corticosteroid.

13. The method of claim 11, wherein said first drug is a tricyclic compound and said second drug is a corticosteroid.

14. The method of claim 13, wherein said first drug is amoxapine and said second drug is prednisolone.

15. The method of claim 11, wherein said first drug is an SSRI and said second drug is a corticosteroid.

16. The method of claim 15, wherein said first drug is paroxetine and said second drug is prednisolone.

17. The method of claim 11, wherein said first drug is a tetra-substituted pyrimidopyrimidine and said second drug is a corticosteroid.

18. The method of claim 17, wherein said first drug is dipyridamole and second drug is prednisolone.

19. The method of claim 11, wherein said first drug is NsIDI and said second drug is an antihistamine.

20. The method of claim 19, wherein said first drug is cyclosporin A and said second drug is loratadine.

21. The method of claim 11, wherein said first drug is a tricyclic compound and said second drug is a corticosteroid.

22. The method of claim 21, wherein said first drug is nortriptyline and said second drug is budesonide.

23. The method of claim 22, wherein said nortriptyline and said budesonide are formulated for inhalation.

24. The method of claim 11, wherein said first drug is a tetra-substituted pyrimidopyrimidine and said second drug is an antihistamine.

25. The method of claim 24, wherein said first drug is dipyridamole and second drug is loratadine.

26. A pharmaceutical composition comprising a unit dosage form comprising a first drug selected from tricyclic compounds, SSRIs, SNRIs, NsIDIs, antihistamines, and tetra-substituted pyrimidopyrimidines; and a second drug formulated for sustained release.

27. The composition of claim 26, wherein said unit dosage form is a bilayer tablet having a first layer comprising said first drug not formulated for sustained release and a second layer comprising said second drug formulated for sustained release.

28. The composition of claim 26, wherein said unit dosage form is a tablet having an inner core comprising said second drug formulated for sustained release and an outer coat comprising said first drug not formulated for sustained release.

29. The composition of claims 27 or 28, wherein said tablet further comprises said second drug not formulated for sustained release.

30. The composition of claim 26, wherein said unit dosage form is a capsule having beads comprising said second drug formulated for sustained release and beads comprising said first drug not formulated for sustained release.

31. The composition of claim 30, wherein said capsule further comprises beads comprising said second drug formulated for sustained release.

32. The composition of claim 26, wherein said first drug is a tricyclic compound and said second drug is a corticosteroid.

33. The composition of claim 32, wherein said first drug is amoxapine and said second drug is prednisolone.

34. The composition of claim 32, wherein said first drug is nortriptyline and said second drug is budesonide.

35. The composition of claim 34, wherein said nortriptyline and said budesonide are formulated for inhalation.

36. The composition of claim 26, wherein said first drug is an SSRI and said second drug is a corticosteroid.

37. The composition of claim 36, wherein said first drug is paroxetine and said second drug is prednisolone.

38. The composition of claim 26, wherein said first drug is a tetra-substituted pyrimidopyrimidine and said second drug is a corticosteroid.

39. The composition of claim 38, wherein said first drug is dipyridamole and said second drug is prednisolone.

40. The composition of claim 26, wherein said first drug is NsIDI and said second drug is an antihistamine.

41. The composition of claim 40, wherein said first drug is cyclosporin A and said second drug is loratadine.

42. The composition of claim 26, wherein said first drug is a tetra-substituted pyrimidopyrimidine and said second drug is an antihistamine.

43. The composition of claim 42, wherein said first drug is dipyridamole and second drug is loratadine.

44. A kit comprising:
(a) a first drug not formulated for sustained release,
(b) a said second drug formulated for sustained release; and
(c) instructions for administering simultaneously, or within 30 minutes of one another, said first drug and said second drug.

45. The kit of claim 44, wherein said first drug is a tricyclic compound and said second drug is a corticosteroid.

46. The kit of claim 45, wherein said first drug is amoxapine and said second drug is prednisolone.

47. The kit of claim 45, wherein said first drug is nortriptyline and said second drug is budesonide.

48. The kit of claim 44, wherein said first drug is an SSRI and said second drug is a corticosteroid.

49. The kit of claim 48, wherein said first drug is paroxetine and said second drug is prednisolone.

50. The kit of claim 44, wherein said first drug is a tetra-substituted pyrimidopyrimidine and said second drug is a corticosteroid.

51. The kit of claim 50, wherein said first drug is dipyridamole and said second drug is prednisolone.

52. The kit of claim 44, wherein said first drug is NsIDI and said second drug is an antihistamine.

53. The kit of claim 52, wherein said first drug is cyclosporin A and said second drug is loratadine.

54. The kit of claim 44, wherein said first drug is a tetra-substituted pyrimidopyrimidine and said second drug is an antihistamine.

55. The kit of claim 54, wherein said first drug is dipyridamole and second drug is loratadine.
